(19) United States
(12) Patent Application Publication
(10) Pub. No.: US 2013/0095501 A1
(43) Pub. Date: Apr. 18, 2013

(54) LEGIONELLA TEST

(71) Applicants: Juergen Bueddefeld, Duisburg (DE); Anna Nickisch-Hartfiel, Krefeld (DE); Jan Consbruch, Krefeld (DE); Peter Klauth, Moenchengladbach (DE)

(72) Inventors: Juergen Bueddefeld, Duisburg (DE); Anna Nickisch-Hartfiel, Krefeld (DE); Jan Consbruch, Krefeld (DE); Peter Klauth, Moenchengladbach (DE)

(73) Assignee: HOCHSCHULE NIEDERRHEIN, KREFELD (DE)

(21) Appl. No.: 13/704,313

(22) PCT Filed: Jun. 6, 2011
(86) PCT No.: PCT/EP2011/059295

§ 371 Date: Dec. 14, 2012

(30) Foreign Application Priority Data
Jun. 16, 2010 (DE) 10 2010 017 396.7

(51) Int. Cl. G01N 33/569

(52) U.S. Cl. CPC G01N 33/56911
USPC 435/7.1; 435/287.2

(57) ABSTRACT

A device and method for qualitatively and/or quantitatively detecting Legionella in water or aqueous solutions comprising at least one inlet for introducing a sample volume into a filtration cell, a filtration device, a device for separating immunomagnetically bonded Legionella, a detection cell, a device for transferring immunomagnetically bonded Legionella into the detection cell, a device for exciting a fluorescence of Legionella having fluorescent marking, and a detector unit for detecting the fluorescence of Legionella having fluorescent marking is provided. The microorganisms present in a sample volume can be filtered out of the sample volume by means of the filtration device in the filtration cell, immunomagnetic compounds for selectively marking Legionella can be introduced into the retentate of the filtration cell, and the immunomagnetically bonded Legionella can be separated from other microorganisms by means of the device and transferred into the detection cell by means of a transfer device.

### FIELD OF TECHNOLOGY

The following relates to an apparatus and a method for qualitatively and/or quantitatively determining Legionellae in water or aqueous solutions.

### BACKGROUND

Legionellae are a genus of rod-shaped bacteria of the family Legionellaceae. They are water-dwelling, Gram-negative, non-spore-forming bacteria which are mobile owing to one or more polar or subpolar flagella. All Legionellae should be considered potentially as pathogenic to humans. Currently, more than 48 species and 70 serogroups are known. The most important species with respect to human diseases is Legionella pneumophila, which is considered to be the pathogen for Legionnaire's disease. Legionellae can occur both in fresh water and in salt water and have optimal living conditions in a temperature range between 25° C. and 50° C. For instance, they frequently occur in installations such as hot-water generation and distribution systems, swimming pools, air washers in air-conditioning systems, cooling towers, dead legs, water tanks and the like. More particularly, they can appear in hot water pipes, or cold water pipes acted on by external heat, which are not used over a prolonged period. This may be the case, for example, in hotels and hospitals with irregular room usage, or else public buildings such as, for example, schools. A further habitat for Legionellae are biofilms, i.e., films composed of a thin slime layer in which microorganisms such as bacteria, fungi or protozoa are embedded and as frequently appear in humid rooms or at interfaces between water surface and a solid surface, for example pipes.

Transmission of Legionellae to living organisms such as humans is possible in principle through contact with tap water or airborne infection if the Legionellae reach the deep sections of the lungs. However, not every contact with Legionellae-containing water leads to a health risk. Disease can result only when bacteria-containing water in the form of an aerosol is inhaled, for example the inhalation of contaminated water during showering, via air-conditioning systems, via lawn sprinklers or in whirlpools.

In the Federal Republic of Germany, the direct or indirect detection of an acute infection due to Legionellae must be reported by the diagnosing laboratory under section 7 of the German Protection Against Infection Act (Infektionsschutzgesetz). Since 2001, such notifiable diseases have been recorded by the Robert Koch Institute in Berlin. In 2004, 475 reported cases of legionellosis were registered by this institute. Out of the cases of Legionnaire's disease which became known, the disease ended fatally for previously healthy individuals in about 15% of cases, and for individuals with immunodeficiency and preexisting heart/lung diseases in up to 70% of cases. The largest outbreak of a Legionella epidemic in Germany and one of the largest worldwide occurred at the start of January 2010 in the Ulm area, with 5 dead and 64 infected, and the source causing this epidemic was traced back to the cooling towers belonging to a combined heat and power plant in the proximity of Ulm central station.

According to the specifications of the German Technical and Scientific Association for Gas and Water (DVGW; Deutscher Verein des Gas- und Wasserfaches e.V.) worksheet W 551, drinking water containing 100 CFU (colony-forming units)/100 ml is considered to be contaminated with a low infection risk. The need for action is thus necessary starting from a value of >10,000 CFU/100 ml, which necessitates immediate measures such as, for example, disinfection of the supply network and the imposition of a temporary ban on usage. Furthermore, the German Drinking Water Ordinance (Trinkwasserverordnung) of 2001 stipulates that all public buildings such as, for example, schools, kindergartens, hospitals, restaurants and other communal facilities must be regularly tested by the local public health department for possible contamination due to Legionellae. Residential buildings and industrial plants can also be included if necessary into such monitoring. A prerequisite therefor is observance of the chemical and physical-technical parameters of the German Drinking Water Ordinance and consideration of the generally acknowledged rules of the art, inter alia, various DIN ordinances, VDE guidelines 6023 and worksheets W 551 and W 553 of the DVGW.

Described by Yanez, M. A., et al. in “Quantitative detection of Legionella pneumophila in water samples by immunomagnetic purification and real-time PCR amplification of the dotA gene”, Applied and Environmental Microbiology, Vol. 71, No. 7, 07, pages 3434-3436, is a method for quantitatively determining Legionella pneumophila in water samples by means of immunomagnetic purification and real-time PCR amplification of dotA genes. Described by Füchslin et al. in “Rapid and quantitative detection of Legionella pneumophila applying immunomagnetic separation and flow cytometry”, Cytometry Part A, 77A, March 2010, pages 264-274, is a method for quantitatively determining Legionella pneumophila in water samples by means of a combination of immunomagnetic separation and flow cytometry.

The prior art to date provides for on-site sampling and detection in a commercial laboratory or public health department laboratory that is equipped for Legionella testing. With these methods, the large amounts of water to be tested, as stipulated in the ordinance valid since 2001, cannot be analyzed problem-free. Furthermore, the tests must be carried out by appropriately trained qualified personnel, and are frequently lengthy and also relatively expensive.

### SUMMARY

It is therefore an object of the present invention to specify an apparatus and a method with which inexpensive and rapid on-site testing for possible contamination with Legionellae is possible without the involvement of qualified personnel.

This object is achieved, with respect to the apparatus, by the subject matter of claim 1 and, with respect to the method, by a method as claimed in claim 10. Embodiments of the invention can be found in the dependent claims and the description which follows.

The invention proposes an apparatus, more particularly a portable apparatus, for qualitatively and/or quantitatively determining Legionellae in water or aqueous solutions, which apparatus comprises at least one intake for introducing a sample volume into a filtration cell, a filtration device, a device for separating immunomagnetically bound Legionellae, a detection cell, a device for transferring immunomagnetically bound Legionellae into the detection cell, a device for exciting fluorescence of fluorescently labeled Legionellae, and a detector unit for detecting the fluorescence of fluorescently labeled Legionellae, wherein the microorganisms contained in a sample volume in the filtration cell are filtratable from the sample volume by means of the filtration device, wherein immunomagnetic labels for selectively labeling Legionellae are introducible into the retentate of the filtration cell, wherein the immunomagnetically bound Legionellae are removable from other microorganisms by means of the device and transferable into the detection cell by means of a transfer device, wherein the transferred immunomagnetically bound Legionellae in the detection cell are labelable with a fluorescent label and fluorescence of the fluorescently labeled Legionellae that is excited by means of the excitation source is detectable by the detector.

With the apparatus according to the invention, it is possible for the first time to carry out the detection, as required under section 3 no. 2 letter c of the German Drinking Water Ordinance 2001, of the absence of Legionellae by means of a preferably mobile and automatic instrument without qualified personnel and without contravening the German Protection Against Infection Act, sections 44 ff.

The apparatus according to the invention works in multiple stages, in which microorganisms contained in a defined sample amount are removed by means of filtration, and the Legionellae are selectively separated from the removed microorganisms by means of immunomagnetic methods in order to be subsequently labeled by means of suitable fluorescent labels. The labeled Legionellae are excited by a suitable excitation source to emit fluorescence, which can subsequently be registered by means of a detector and qualitatively and/or quantitatively evaluated by means of a computer system. The apparatus according to the invention is particularly suitable for temporary or permanent integration into aquiferous systems, for example: drinking water supply network, cooling towers, air-conditioning systems, etc.

In stage 1, the apparatus is connected to a water supply to be tested for possible contamination with Legionellae, for example by means of a standard thread or quick coupling (Gardena connector). The discharge from the apparatus is connected to a drain. Solenoid valves and a downstream flow meter then allow a predefined amount of water, for example 10 L, to flow through the apparatus according to the invention.

In stage 2, the water flows through an ultrafiltration cell or a retentostat, having for example a cell volume of 300 ml. A stirrer can be provided above the filter membrane, which has a pore size of 0.2 μm for example, in order to keep the microorganisms in the volume of the filter cell in suspension. A defined amount of sample liquid, for example 10 L, drains across the water-permeable filter membrane; the contained microorganisms are retained and concentrated. After flow-through of the predefined volume, the solenoid valves are closed, and so-called immunomagnetic beads (e.g., Dynabeads MAX Legionella) are added to the filter cell, which beads accumulate via their antibody segments on the Legionellae. A series of experiments with immunomagnetic beads have shown that even one single cell can be reliably removed from a sample volume of, for example, 300 mL. Owing to the high permissible concentration of Legionellae in drinking water (<100/100 mL) and to the filtration of large sample volumes that is provided according to the invention, it is possible in the aforementioned example to reliably separate the theoretically permissible 10,000 Legionellae (concentrated in 300 mL) from 10 L of water and to supply them to a detection according to the invention.

In stage 3, a “magnetic finger”, which can, for example, consist of a plastic shell having inner neodymium-boron-iron magnets, is used to bind the immunomagnetic beads and hence the Legionellae accumulated thereon to the plastic outer shell. Alternatively, the magnetic field required for accumulating the immunomagnetically labeled Legionellae can be produced by an electromagnet.

In stage 4, the magnetic finger is moved out of the filter cell, for example by means of a servo-controlled arm, and transferred into a detection cell, for example a 0.5 mL Eppendorf cuvette. The inner magnet of the magnetic finger is removed, for example by means of a servo-controlled iron wire which is guided into the interior of the magnetic finger, and the plastic outer shell is rinsed, for example with 100 μl of Ringer's solution, with the rinse liquid flowing into the detection cell. As a result, the adherent immunomagnetically labeled Legionellae are transferred into the detection cell. In one embodiment of the apparatus according to the invention using an electromagnet of the immunomagnetically bound Legionellae on a magnetic finger, the power supply to the electromagnet can simply be interrupted in order to detach the bound Legionellae.

In stage 5, a fluorescent label, for example 10 μl of a SYBR Green solution 1/1000 stock, is added to the solution located in the detection cell. The content of the detection cell is then excited by means of a suitable excitation source, for example a diode laser, to emit fluorescence and this is measured. The detector used here can, for example, be an avalanche diode or an array of avalanche diodes, though all other types of suitable detectors, for example photomultipliers or CCD sensors, can also be used. In one embodiment of the apparatus according to the invention, the detector unit has an avalanche diode or an array of avalanche diodes, more particularly a linear array. Such arrays are also known as multi-pixel photon counters (MPPC, from Hamamatsu). Avalanche diodes utilize the so-called avalanche effect. Accelerated by an adequately adjusted external field strength, electrons excited by the incident light have a sufficiently high energy that, after a collision with a valence electron, they not only make this electron-hole pair available as charge carrier in the conduction band, but themselves do not recombine and continue to remain in the conduction band, where they can again produce free charge carriers. As a result, the number of free charge carriers in the conduction band increases like an avalanche and exponentially. An advantage of using avalanche diodes in the detector units of an apparatus according to the invention is that even little light quanta appearing on the detector units lead to a highly detectable signal. Individual avalanche diodes may be “blind” to further events for a brief period after the induction of an avalanche (Geiger mode), and so any photons following in rapid succession are not recognized. This is circumvented by arrays of avalanche diodes, for example the abovementioned MPPCs, which exploit the low probability of multiphoton events, i.e., multiple photons at the same time at the same place, by means of a high number of individual diode pixels in a micrometer grid.

The amount of emitted fluorescent light is proportional to the amount of DNA and thus to the amount of Legionellae. The signal of the MPPC photodetector, which is used for example, can then be converted into a Legionella concentration in a 100 mL volume by means of an externally recorded standard curve, as required according to DVGW worksheet W551.

In an optional stage 6, the Legionellae in the apparatus can be killed by addition of a suitable disinfectant or by other measures such as heating of the contaminated regions of the apparatus to a sufficiently high temperature (e.g., >50° C.) or irradiation with UV light.

In one embodiment of the invention, the excitation source used for the fluorescence is a diode laser having a radiation wavelength of from 340 nm to 520 nm, preferably from 365 nm to 488 nm, more particularly 365 nm or 405 nm. Such diode lasers are advantageously long-lasting as standardized electronic components. For example, blue-emitting diode lasers having a wavelength of 405 nm are installed in Blue-Ray® players as scanning laser and are therefore easily and inexpensively available on the market.

In a further embodiment of the invention, it has a separation cell into which the filtered microorganisms are transferred and in which the immunomagnetic separation of the Legionellae from the other filtered microorganisms takes place.

In a further embodiment of the invention, the immunomagnetic separation and the subsequent fluorescent labeling and detection of the fluorescence take place in a common cell, and so separation cell and detection cell coincide. In such an embodiment, the immunomagnetically labeled Legionellae can be held by the magnetic finger and the other microorganisms can be rinsed out. Subsequently, a suitable fluorescent-label solution is added to the cell and the abovedescribed excitation sources excite fluorescence, which can then be detected.

In a further embodiment of the apparatus according to the invention, at least the filtration cell, the device for separation, the detection cell and the transfer device are arranged in a closed housing. For the purposes of the invention, closed housing is to be understood to mean one which prevents escape of Legionellae into the environment.

In a particularly preferred embodiment of the invention, the apparatus is designed as a transportable apparatus, and so it can be set up in a mobile manner at different operational sites.

In a further embodiment of the invention, it has an integrated microprocessor unit which controls the apparatus and on which evaluation of the detected fluorescence signals takes place.

In a further embodiment of the invention, it has a communication device, for example a network interface or a GSM module or the like, by means of which the apparatus can be remotely controlled and/or via which the apparatus can transmit information, for example operational states or measured results.

In a further embodiment, the apparatus according to the invention has a user interface via which the apparatus can be adjusted and/or measured results can be read.

With respect to the method, the object of the invention is achieved by a method for qualitatively and/or quantitatively determining Legionellae in water or aqueous solutions, comprising at least the method steps of:
introducing a defined sample volume into a filtration cell;
removing the microorganisms contained in the sample volume by means of ultrafiltration;
transferring the removed microorganisms into a separation cell;
selectively immunomagnetically labeling the Legionellae contained in the removed microorganisms;
separating the immunomagnetically labeled Legionellae;
transferring the separated immunomagnetically labeled Legionellae into a detection cell;
labeling the transferred Legionellae with a fluorescent label, preferably SYBR Green;
exciting the fluorescence of the fluorescently labeled Legionellae with an excitation source, preferably a laser, particularly preferably a diode laser, having a radiation wavelength of from 340 nm to 520 nm, preferably 365 nm or 405 nm.

The method according to the invention is particularly suitable for implementation in an apparatus according to the invention.

Owing to the combination according to the invention of filter cell for accumulation without culturing with downstream immunoseparation and fluorescence measurement, it is especially possible to create an apparatus in the form of a mobile and stand-alone instrument for qualitatively and/or quantitatively determining Legionellae. The use of MPPC detectors makes it possible to carry out the recording and evaluation of measured values by means of standard microprocessors. This has a positive effect on the complexity of the detection method and thus on its costs. A further advantage is provided by the increasingly inexpensive blue-emitting laser diodes or UV diode lasers.

### BRIEF DESCRIPTION

The invention will now be more particularly elucidated by means of figures and exemplary embodiments, but without restricting the invention to said exemplary embodiments.

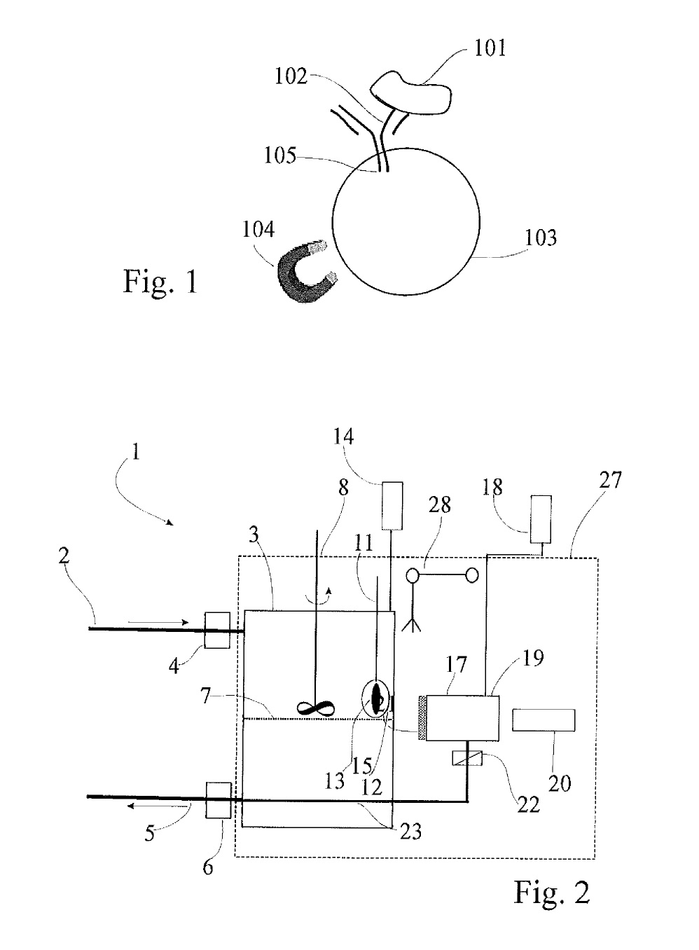

FIG. 1 shows the diagrammatic representation of an immunomagnetic bead;

FIG. 2 shows the diagrammatic structure of one embodiment of an apparatus according to the invention;

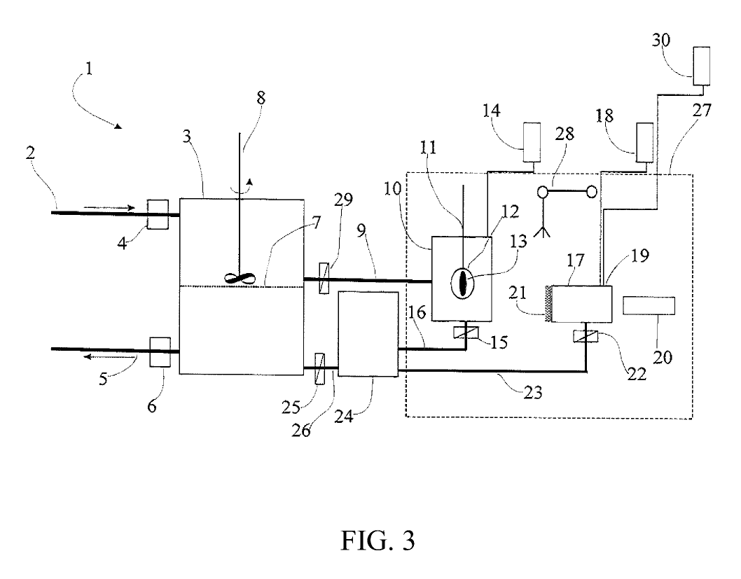

FIG. 3 shows the diagrammatic structure of a further embodiment of an apparatus according to the invention.

### DETAILED DESCRIPTION

FIG. 1 shows the functional principle of an immunomagnetic bead. A microorganism 101, for example a Legionella, is bound to a particle 103 via a specific antibody 102 or antibody segment. The antibody 102 is connected to the particle 103 via appropriate binding sites 105. By means of its specific binding to epitopes particular microorganisms, a microorganism can thus be bound with high specificity to a particle 103. The particle 103 (bead) can be of differing configuration. In the case of the present invention, the particles 103 are magnetic and can thus be bound to a magnet 104. This makes possible magnetic removal of the particles 103 and thus likewise removal of the Legionellae 101 bound to the particles 103 via the antibodies 102.

FIG. 2 shows one embodiment of an apparatus 1 according to the invention. A sample volume is supplied to a filtration cell 3 via an inlet 2. The filtration cell 3 has a filtration device 7, for example a membrane filter having a pore size of 0.2 μm. The permeate can leave the filtration cell 3 via an outlet 5. Flow meters and valves 4, 6, preferably solenoid valves, are provided in the intake 2 and/or the outlet 5 in order to interrupt the intake of sample volume when a predefinable volume has been reached. A stirring device 8 is arranged above the filtration device in order to keep the microorganisms contained in the retentate in suspension. After a predefined sample volume, for example 10 L, has been reached, the intake 2 is closed by the solenoid valve 4. A sufficient amount (10⁵) of immunomagnetic beads specific for Legionellae (e.g., Dynabeads® MAX Legionella from Invitrogen) is added to the retentate from a reservoir 14. After an appropriate reaction time (30 min), the immunomagnetically labeled Legionellae are separated from the other microorganisms located in the retentate by means of a separation device 11. In the embodiment shown, the separation device is formed as a “magnetic finger” which consists of a plastic shell 12 having inner neodymium-boron-iron magnets 13, and the immunomagnetic beads are therefore bound to the plastic outer shell 12. This process lasts about 30 minutes, while the stirring device 8 stirs further. Subsequently, the separated Legionellae are transferred by transferring the magnetic finger 11 into a detection cell 17. This is achieved by means of a transfer device 28, which is formed as a servo arm in the example shown. In the detection cell 17, which is formed by a 0.5 mL Eppendorf cuvette in the example shown, the inner magnet 13 is removed by means of an iron wire moved by a servo, which wire is guided into the interior of the magnetic finger. The servo arm of the transfer device 28 serves as the manipulator for guiding the iron wire. The plastic outer shell 12 is rinsed with 100 μl of Ringer's solution from storage container 30, and the rinse liquid flows into the detection cell 17. A fluorescent label, in this case a SYBR Green solution (1/1000 stock, 10 μL), is supplied to the detection cell 17 from reservoir 18 and the content of the detection cell 17 is stirred for 10 min with the plastic outer shell. Subsequently, the fluorescence is excited by means of an excitation source 20, in this case a diode laser having a wavelength of 375 nm or 473 nm. The emission wavelength of the system thus excited to emit fluorescence is 545 nm, which is detected by means of the detection unit 21, in this case an MPPC sensor. A bandpass filter for 545/10 nm is placed before the detector. The amount of emitted fluorescent light is proportional to the amount of Legionellae. One Legionella DNA has 3.4×10⁶ base pairs. SYBR Green fluoresces most intensely at 10 molecules per base pair. The threshold value for Legionellae is 10⁴. Therefore, in the fluorescence, 3.4×10⁶×10×10⁴=3.4×10¹¹ photons are emitted, minus the following losses: Quantum yield 50%; losses in the detection owing to the spherical propagation of the fluorescence 90%=3.4×10¹¹×0.5×0.1=1.7×10¹⁰ photons per fluorescence cycle, i.e., in 1 μs. By integration of the MPPC module to 1 ms, 1.7×10¹⁰ photons×1000 μs=1.7×10¹³ photons/ms are then obtained. The dark noise is 10⁶ photons/ms at room temperature. The remaining residual dynamic range of 10⁷ is sufficient for reliable detection of a Legionella threshold value. To avoid environmental contamination with Legionellae, at least the filtration cell 3 and the detection cell 17 are accommodated in a housing which is closed and impermeable to microorganisms. After completion of the measurement, the Legionellae located in the apparatus are killed by addition of a disinfectant and/or thermal disinfection and/or UV disinfection. For this purpose, the apparatus has appropriate devices such as storage containers for disinfection solutions, heating devices and UV lamps.

FIG. 3 shows a further embodiment of an apparatus 1 according to the invention. In the embodiment shown, the apparatus 1 according to the invention has a separation cell 10 which is separate from the filtration cell 3 and into which the retentate of the filtration is transferred via a transfer line 9 controllable by means of a solenoid valve 29. The immunomagnetic labeling and separation then take place in the separation cell 10 in the manner previously described in relation to FIG. 1. After the immunomagnetic separation, the residue which is located in the separation cell and which is contaminated with the further microorganisms which had been filtered out is transferred via a transfer line 16 controllable by means of a solenoid valve 15 into a disinfection chamber 24, in which the microorganisms contained in the residue are killed by means of the measures already described in relation to FIG. 1. After the fluorescence measurement, the sample which is located in the detection cell and which is contaminated with the Legionellae is transferred via a transfer line 23 controllable by means of a solenoid valve 22 into the disinfection chamber 24, in which the microorganisms contained in the residue are killed by means of the measures already described in relation to FIG. 1. By opening a solenoid valve 25, the decontaminated residue can then be discharged into the outlet 5 via line 26. In such an embodiment, at least the separation cell 10, the detection cell 17 and the transfer device 28 are arranged in a closed housing.

1. A portable apparatus for qualitatively and/or quantitatively determining Legionellae in water or aqueous solutions, comprising:
at least one intake for introducing a sample volume into a filtration cell, a filtration device, a device for separating immunomagnetically bound Legionellae, a detection cell, a device for transferring immunomagnetically bound Legionellae into the detection cell, a device for exciting fluorescense of fluorescently labeled Legionellae, and a detector for detecting the fluorescence of fluorescently labeled Legionellae;
wherein the microorganisms contained in the sample volume in the filtration cell are filtratable from the sample volume by means of the filtration device,
wherein immunomagnetic bonds for selectively labeling Legionellae are introducible into a retentate of the filtration cell,
wherein the immunomagnetically bound Legionellae are removable from other microorganisms by means of the device and transferable into the detection cell by means of a transfer device, further wherein the transferred immunomagnetically bound Legionellae in the detection cell are labelable with a fluorescent label and fluorescence of the fluorescently labeled Legionellae that is excited by means of the excitation source is detectable by the detector.

2. The apparatus as claimed in claim 1, wherein the filtration cell has a membrane filter as the filtration device.

3. The apparatus as claimed in claim 1, wherein the device for separating immunomagnetically labeled Legionellae has an external shell and a magnet arranged in said shell.

4. The apparatus as claimed in claims claim 1, further comprising a device for killing microorganisms located in the apparatus after qualitative and/or quantitative Legionella determination.

5. The apparatus as claimed in claim 1, further comprising a stirrer in the filtration cell above the filtration device.

6. The apparatus as claimed in claim 1, further comprising a standard threaded connector and/or a standard garden connector as intake.

7. The apparatus as claimed in claim 1, wherein the detector has an MPPC sensor.

8. The apparatus as claimed in claim 1, wherein the excitation source has by means of a diode laser having a wavelength of from 340 nm to 520 nm, preferably from 365 nm to 488 nm, more particularly 365 nm or 405 nm.

9. A method for qualitatively and/or quantitatively determining Legionellae in water or aqueous solutions, comprising at least the method steps of:
introducing a defined sample volume into a filtration cell;
removing the microorganisms contained in the defined sample volume by means of ultrafiltration;
transferring the removed microorganisms into a separation cell;
selectively immunomagnetically binding the Legionellae contained in the removed microorganisms;
separating the immunomagnetically bound Legionellae;
transferring the separated immunomagnetically bound Legionellae into a detection cell;
labeling the transferred Legionellae with a fluorescent label;
exciting the fluorescence of the fluorescently labeled Legionellae with an excitation source having a radiation wavelength of from 340 nm to 520 nm;
detecting the excited fluorescence by means of a detector; and
determining the Legionella concentration in the sample volume on the basis of the measured fluorescence.

10. The method of claim 9, wherein the radiation wavelength is 365 nm to 488 nm.

11. The method of claim 9, wherein the radiation wavelength is 365 nm, 375 nm, 405 nm or 473 nm.

12. The method of claim 9, wherein the excitation source is a diode laser.

13. The method of claim 9, wherein the sensor is an MPPC sensor.

14. The method of claim 9, wherein the fluorescent label is SYBR Green.
